(19) United States
(12) Patent Application Publication
(10) Pub. No.: US 2006/0025463 A1
(43) Pub. Date: Feb. 2, 2006

(54) Enoximone sulfoxide enantiomers and their use in the treatment of PDE-III mediated diseases

(76) Inventors: Michael Bristow, Englewood, CO (US); Michael J. Gerber, Denver, CO (US); Rick Gorczynski, Westminster, CO (US)

Correspondence Address: FULBRIGHT & JAWORSKI L.L.P., 600 CONGRESS AVE., SUITE 2400, AUSTIN, TX, 78701, US

(21) Appl. No.: 11/087,059

(22) Filed: Mar. 22, 2005

Related U.S. Application Data
(60) Provisional application No. 60/555,261, filed on Mar. 22, 2004.

(51) Int. Cl. A61K 31/4166

(52) U.S. Cl. 514/389

(57) ABSTRACT

The present invention provides the (S)-(−) enoximone sulfoxide enantiomer, as well as pharmaceutical formulations of the purified (S)-(−) sulfoxide enantiomer. Also provided are methods of treating diseases in which inhibition of PDE-III may be beneficial.

This application claims benefit of priority to U.S. Provisional Application Ser. No. 60/555,261 filed Mar. 22, 2004, the entire contents of which are hereby incorporated by reference.

### BACKGROUND OF THE INVENTION

1. Field of the Invention

The present invention relates generally to the fields of cardiology and medicine. More particularly, it concerns pure enantiomeric formulations of enoximone sulfoxide for use in treating cardiovascular diseases, heart failure, and a variety of diseases where inhibition of phosphodiesterase-III (PDE-III) would be beneficial.

2. Description of Related Art

Phosphodiesterases (PDEs) are a class of intracellular enzymes involved in the metabolism of the second messenger nucleotides, cyclic adenosine monophosphate (cAMP), and cyclic guanosine monophosphate (cGMP) (see, Doherty, “Oral, Transdermal and Transurethral Therapies for Erectile Dysfunction” in Male Infertility and Dysfunction, Hellstrom, ed., Chapter 34 (New York, N.Y.: Springer-Verlag, 1997)). Numerous phosphodiesterase inhibitors have previously been described in the literature for a variety of therapeutic uses, including treatment of obstructive lung disease, allergies, hypertension, angina, congestive heart failure and depression (see, Goodman and Gilman's The Pharmacological Basis of Therapeutics Tenth Edition, Chapter 34). Oral and parenteral administration of PDE-V inhibitors, as alluded to above, have also been used for the treatment of erectile dysfunction (Doherty, supra; see also PCT Publication Nos. WO 96/16644 and WO 94/28902).

As explained by Komas et al. (1996), those initially working in the field partially purified what was believed to be a single enzyme responsible for specifically hydrolyzing the 3′- bond of cyclic nucleotides. However, it later became clear that multiple forms of phosphodiesterase inhibitors were present in different tissues; the enzymes were classified into three major groups, one of which exhibited high affinity for cAMP and designated as the “low Kₘ” cAMP PDE. This “low Kₘ” cAMP PDE was ultimately discovered to consist of two distinct isoenzymes having entirely different properties, including physical properties, kinetic characteristics and inhibitor specificities. One isoenzyme was found to be very sensitive to inhibition by cilostamide and cGMP, and is now known as the cAMP-specific, cGMP-inhibited cyclic nucleotide phosphodiesterase (cGI-PDE) or PDE III, while the second isoenzyme was classified as PDE IV (Komas et al., 1996).

The phosphodiesterases have now been classified into ten major families, Types I- X, based on amino acid or DNA sequences. The members of the family vary in their tissue, cellular and subcellular distribution, as well as their links to cAMP and cGMP pathways. For example, the corpora cavernosa contains: Type III phosphodiesterases, which as explained above are cAMP-specific cGMP inhibitable; Type IV phosphodiesterases, the high affinity, high-specificity cAMP-specific form; and Type V phosphodiesterases, one of the cGMP-specific forms.

Various compounds are known as inhibitors of phosphodiesterases, including vinpocetine, milrinone, amrinone, pimobendan, cilostamide, enoximone, piroximone, vesnarinone, rolipram, RO20-1724, zaprinast, dipyridamole, pentoxifylline, sildenafil citrate (Viagra[R]), doxazosin, papaverine, prazosin, terazosin, trimazosin and hydralazine. PCT Publication No. WO 94/28902 discloses a series of pyrazole [4,3-d] pyrimidin-7-ones cGMP phosphodiesterase inhibitors. PCT Publication No. WO 96/16644 also discloses a variety of cGMP phosphodiesterase inhibitors, including griseolic acid derivatives, 2-phenylpurinone derivatives, phenylpyridone derivatives, fused and condensed pyrimidines, a pyrimdopyrimidine derivative, a purine compound, a quinazoline compound, a phenylpyrimidone derivative, an imidazoquinoxalinone derivative or aza analogues thereof, a phenylpyridone derivative, and others.

PDE-III has been implicated as a target molecule for therapy in a variety of diseases, including a variety of cardiovascular diseases. Cardiac hypertrophy, for example, is one such disease for which inhibition of PDE-III is indicated. Cardiac hypertrophy is an adaptive response of the heart to many forms of cardiac disease, including hypertension, mechanical load abnormalities, myocardial infarction, valvular dysfunction, certain cardiac arrhythmias, endocrine disorders and genetic mutations in cardiac contractile protein genes. While the hypertrophic response is thought to be an initially compensatory mechanism that augments cardiac performance, sustained hypertrophy is maladaptive and frequently leads to ventricular dilation and the clinical syndrome of heart failure. Accordingly, cardiac hypertrophy has been established as an independent risk factor for cardiac morbidity and mortality (Levy et al., 1990).

Treatment with pharmacological agents represents the primary mechanism for reducing or eliminating the manifestations of heart failure. Diuretics constitute the first line of treatment for mild-to-moderate heart failure. Unfortunately, many of the commonly used diuretics (e.g., the thiazides) have numerous adverse effects. For example, certain diuretics may increase serum cholesterol and triglycerides. Moreover, diuretics are generally ineffective for patients suffering from severe heart failure. If diuretics are ineffective, vasodilatory agents may be used; the angiotensin converting (ACE) inhibitors (e.g., enalopril and lisinopril) not only provide symptomatic relief, they also have been reported to decrease mortality (Young et al., 1989). Again, however, the ACE inhibitors are associated with adverse effects that result in their being contraindicated in patients with certain disease states (e.g., renal artery stenosis). Similarly, inotropic agent therapy (i.e., a drug that improves cardiac output by increasing the force of myocardial muscle contraction) is associated with a panoply of adverse reactions, including gastrointestinal problems and central nervous system dysfunction.

Thus, many of the currently used pharmacological agents have severe shortcomings in particular patient populations. The availability of new, safe and effective agents, such as PDE-III inhibitors, would undoubtedly benefit patients who either cannot use the pharmacological modalities presently available, or who do not receive adequate relief from those modalities.

### SUMMARY OF THE INVENTION

Thus, in accordance with the present invention, there is provided a compound of the formula I 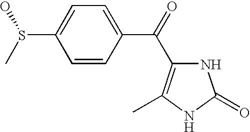
as a pure (S)-(−) enantiomer of the sulfoxide of the pharmaceutical enoximone. In further embodiments of the invention the compound is greater than 70% pure, greater than 75% pure, greater than 80% pure, greater than 85% pure, greater than 90% pure, greater than 95% pure, greater than 97% pure, greater than 98% pure, or greater than 99% pure. In these embodiments it is contemplated that the (S)-(−) form is substantial free of contamination by the (R)-(+) enantiomer.

In one embodiment of the invention there is provided a pharmaceutical comprising the compound of formula I, and all pharmaceutically acceptable salts thereof.

In further embodiments, it is contemplated that the pharmaceutical formulation will be delivered via rapid release, timed release, delayed release, sustained release, oral suspension, parenteral delivery, as a suppository, via intravenous administration, intramuscular administration, intraperitoneally, sublingually, transdermally, or via a nasopharygeal route. Also contempled are solid and liquid forms of the pharmaceutical formulation.

In yet further embodiments of the invention, it is contemplated that the compound will be formulated as an uncoated tablet, a capsule, a powder, a troche, a granule, a liposome, a suppository, a solution, a colloid, an ointment, a cream, a vapor, a spray, a nanoparticle, an inhalant, a nasal solution, an intravenous admixture, an epidermal solution, a buccal table, a syrup, a cream, a lotion, a gel, an emulsion, or an elixir. The formulations may further comprise one or more of a tablet binder, filler, preservative, tablet disintegrant, flow regulator, plasticizer, wetting agent, dispersant, an emulsifier, a solvent, release-slowing agent, an antioxidant, or a propellant gas.

In certain embodiments of the invention, it is contemplated that the formulation will be used to treat a disease state in a patient where inhibition of PDE-III is considered beneficial by administering the pharmaceutical formulation to said patient. The disease state may be selected from the list comprising acute heart failure, chronic heart failure, hemodynamic failure, chronic heart disease, cardiac hypertrophy, platelet disorder, renal disease, renal failure, pulmonary hypertension, PAH, stable angina, unstable angina, erectile dysfunction, myocardial infarction, peripheral vascular disease, asthma, bronchospastic lung disease, chronic obstructive lung disease, gastrointestinal disorders, hypercoagulation states, thrombocytosis, eclampsia, or pre-eclampsia.

It is further contemplated that a second pharmaceutical may be added as a second therapy in addition to the formulation of the present invention. The second pharmaceutical may be selected from the list comprising “beta blockers,” anti- hypertensives, cardiotonics, anti-thrombotics, vasodilators, hormone antagonists, endothelin receptor antagonists, cytokine inhibitors/blockers, calcium channel blockers, other phosphodiesterase inhibitors, or angiotensin type 2 antagonists. The second pharmaceutical may also be the drug ambrisentan or darusentan.

### DESCRIPTION OF ILLUSTRATIVE EMBODIMENTS

Enoximone, in an i.v. formulation, has been used to treat congestive heart failure and to treat patients in cardiac post-surgery or transplant settings. It is a member of a unique chemical class of drugs called imidazolone derivatives and possesses both positive inotropic and vasodilating properties. These dual actions are evidenced clinically by increased contractility plus reduced preload and afterload, resulting in increased cardiac output, with little or no effect on myocardial oxygen consumption. The molecular basis for these effects is the apparent inhibitory action of enoximone on PDE- III, which results in an increase in intracellular levels of cAMP and the consequent inotropic effect. Unfortunately, the i.v. therapy typically requires participation of trained medical personnel, often in a hospital setting. Patient compliance also becomes an issue on self-medication. Enoximone is currently available as a solid dosage drug that may be used as a treatment for heart failure, but it is not yet an approved pharmaceutical and thus, additional drugs that could be used to treat heart failure and related conditions where inhibition of PDE-III would be beneficial would be highly desirable. Enoximone is eliminated from the body both unchanged and after biotransformation. Enoximone sulfoxide is the main metabolite found in man and occurs as a first transformation after ingestion. Enoximone sulfoxide also possesses cardiotonic activity and is a chiral molecule. Enantiomerically pure enoximone sulfoxide is a new compound of the present invention, which provides new compounds and their formulations that may be used for the treatment of heart failure as well as any disease state in which inhibition of PDE-III would be beneficial.

### I. Heart Failure

Heart failure is one of the leading causes of morbidity and mortality in the world. In the U.S. alone, estimates indicate that 3 million people are currently living with cardiomyopathy and another 400,000 are diagnosed on a yearly basis. Dilated cardiomyopathy (DCM), also referred to as “congestive cardiomyopathy,” is the most common form of the cardiomyopathies and has an estimated prevalence of nearly 40 per 100,000 individuals (Durand et al., 1995). Although there are other causes of DCM, familiar dilated cardiomyopathy has been indicated as representing approximately 20% of “idiopathic” DCM. Approximately half of the DCM cases are idiopathic, with the remainder being associated with known disease processes. For example, serious myocardial damage can result from certain drugs used in cancer chemotherapy (e.g., doxorubicin and daunoribucin), or from chronic alcohol abuse. Peripartum cardiomyopathy is another idiopathic form of DCM, as is disease associated with infectious sequelae. In sum, cardiomyopathies, including DCM, are significant public health problems.

Heart disease and its manifestations, including coronary artery disease, myocardial infarction, congestive heart failure and cardiac hypertrophy, clearly present a major health risk in the United States today. The cost to diagnose, treat and support patients suffering from these diseases is well into the billions of dollars. Two particularly severe manifestations of heart disease are myocardial infarction and cardiac hypertrophy. With respect to myocardial infarction, typically an acute thrombocytic coronary occlusion occurs in a coronary artery as a result of atherosclerosis and causes myocardial cell death. Because cardiomyocytes, the heart muscle cells, are terminally differentiated and generally incapable of cell division, they are generally replaced by scar tissue when they die during the course of an acute myocardial infarction. Scar tissue is not contractile, fails to contribute to cardiac function, and often plays a detrimental role in heart function by expanding during cardiac contraction, or by increasing the size and effective radius of the ventricle, for example, becoming hypertrophic.

### II. PDE-III

A. Phosphodiesterases

Phosphodiesterases are enzymes that catalyze the degradation of the cyclic nucleotides, cyclic AMP and cyclic GMP, to the corresponding 5′ nucleotide monophosphates. Ten different phosphodiesterase families have been described to date. These enzymes exist as homodimers and there is structural similarity between the different families. However, they differ in several respects like selectivity for cyclic nucleotides, sensitivity for inhibitors and activators, physiological roles and tissue distribution. Interest in these enzymes has increased of late, both within the medical community and in the general public, as a consequence of sildenafil (Viagra), the medication recently introduced for the treatment of erectile dysfunction. Sildenafil mediates its effects by inhibiting PDE-V, a close relative of PDE-III. Other functions that are mediated by the phosphodiesterases explain visual disturbances, flushing and decreased blood pressure that are some of the side effects seen with PDE-III inhibitors.

B. PDE-III Inhibition

1. Cardiopulmonary Diseases

PDE-III inhibition has been accomplished with drugs known as positive inotropes. Positive inotropic drugs have various mechanisms of action and act differently from many drugs used previously to treat cardiac diseases. Long-term use of cyclic adenosine monophosphate (cAMP)-dependent drugs has adverse effects on the prognosis of heart failure patients, whereas digoxin has a neutral effect on mortality. There are, however, little data on the effects of inotropic drugs on the outcome of patients. Intravenous inotropic agents have been used to treat cardiac emergencies and refractory heart failure. β-Adrenergic agonists are rapid acting and easy to titrate, with short elimination half-life; however, they increase myocardial oxygen consumption and are thus hazardous during myocardial ischaemia. Furthermore they may promote myocyte apoptosis. Phosphodiesterase (PDE) III inhibiting drugs such as enoximone increase contractility by reducing the degradation of cAMP. In addition, they reduce both preload and afterload via vasodilation. Short-term use of intravenous milrinone, another PDE-III inhibitor, has not been associated with increased mortality, and some symptomatic benefit might be obtained when a PDE-III inhibitor is used in refractory heart failure. Furthermore, PDE III inhibitors facilitate weaning from the cardiopulmonary bypass machine after cardiac surgery. The pharmacokinetics of inotropic drugs might sometimes greatly modify and prolong the response to therapy, for example because of long-acting active metabolites. These drugs display considerable differences in their pharmacokinetics and pharmacodynamics, and the selection of the most appropriate inotropic drug should be based on careful consideration of the clinical status of the patient and on the pharmacology of the drug.

PDE profiles of human cell preparations and tissues have also been analyzed by a semiquantitative method using selective PDE inhibitors and activators. Lymphocytes, alveolar macrophages and endothelial cells contain PDE III, and it has been demonstrated that both PDE III and PDE IV have to be inhibited for complete suppression of either tumour necrosis factor-alpha (TNF-alpha) release from macrophages, or lymphocyte proliferation (PDE III/IV cells). PDE inhibitors have been able to inhibit PDE isoenzyme activities and functions of inflammatory cells with potency (Schudt et al., 1995), and thus, PDE-III inhibitors like enoximone may be beneficial in the treatment of pulmonary or asthmatic diseases.

a. Heart Failure and Hypertrophy

Heart disease and its manifestations, including coronary artery disease, myocardial infarction, congestive heart failure and cardiac hypertrophy, clearly presents a major health risk in the United States today. The cost to diagnose, treat and support patients suffering from these diseases is well into the billions of dollars. One particularly severe manifestation of heart disease is cardiac hypertrophy. Regarding hypertrophy, one theory regards this as a disease that resembles aberrant development and, as such, raises the question of whether developmental signals in the heart can contribute to hypertrophic disease. Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to DCM, heart failure, and sudden death. In the United States, approximately half a million individuals are diagnosed with heart failure each year, with a mortality rate approaching 50%.

The causes and effects of cardiac hypertrophy have been extensively documented, but the underlying molecular mechanisms have not been fully elucidated. Understanding these mechanisms is a major concern in the prevention and treatment of cardiac disease and will be crucial as a therapeutic modality in designing new drugs that specifically target cardiac hypertrophy and cardiac heart failure. The symptoms of cardiac hypertrophy initially mimic those of heart failure and may include shortness of breath, fatigue with exertion, the inability to lie flat without becoming short of breath (orthopnea), paroxysmal nocturnal dyspnea, enlarged cardiac dimensions, and/or swelling in the lower legs. Patients also often present with increased blood pressure, extra heart sounds, cardiac murmurs, pulmonary and systemic emboli, chest pain, pulmonary congestion, and palpitations. In addition, DCM causes decreased ejection fractions (i.e., a measure of both intrinsic systolic function and remodeling). The disease is further characterized by ventricular dilation and grossly impaired systolic function due to diminished myocardial contractility, which results in dilated heart failure in many patients. Affected hearts also undergo cell/chamber remodeling as a result of the myocyte/myocardial dysfunction, which contributes to the “DCM phenotype.” As the disease progresses so do the symptoms. Patients with DCM also have a greatly increased incidence of life-threatening arrhythmias, including ventricular tachycardia and ventricular fibrillation. In these patients, an episode of syncope (dizziness) is regarded as a harbinger of sudden death.

Diagnosis of hypertrophy typically depends upon the demonstration of enlarged heart chambers, particularly enlarged ventricles. Enlargement is commonly observable on chest X-rays, but is more accurately assessed using echocardiograms. DCM is often difficult to distinguish from acute myocarditis, valvular heart disease, coronary artery disease, and hypertensive heart disease. Once the diagnosis of dilated cardiomyopathy is made, every effort is made to identify and treat potentially reversible causes and prevent further heart damage. For example, coronary artery disease and valvular heart disease must be ruled out. Anemia, abnormal tachycardias, nutritional deficiencies, alcoholism, thyroid disease and/or other problems need to be addressed and controlled.

As mentioned above, treatment with pharmacological agents still represents the primary mechanism for reducing or eliminating the manifestations of heart failure. Diuretics constitute the first line of treatment for mild-to-moderate heart failure. Unfortunately, many of the commonly used diuretics (e.g., the thiazides) have numerous adverse effects. For example, certain diuretics may increase serum cholesterol and triglycerides. Moreover, diuretics are generally ineffective for patients suffering from severe heart failure.

If diuretics are ineffective, vasodilatory agents may be used; the angiotensin converting (ACE) inhibitors (e.g., enalopril and lisinopril) not only provide symptomatic relief, they also have been reported to decrease mortality (Young et al., 1989). Again, however, the ACE inhibitors are associated with adverse effects that result in their being contraindicated in patients with certain disease states (e.g., renal artery stenosis). Similarly, inotropic agent therapy (i.e., a drug that improves cardiac output by increasing the force of myocardial muscle contraction) has previously been associated with a panoply of adverse reactions, including gastrointestinal problems and central nervous system dysfunction.

Thus, the currently used pharmacological agents have severe shortcomings in particular patient populations. The availability of new, safe and effective agents would undoubtedly benefit patients who either cannot use the pharmacological modalities presently available, or who do not receive adequate relief from those modalities.

b. Hypertension

Pulmonary artery hypertension is a secondary event often caused by cardiac disorders, pulmonary disorders such as COPD or both in combination. Although extremely common, the incidence of pulmonary hypertension has not been accurately determined due in part to the fact that many patients are undiagnosed. As an indicator however, in individuals older than 50 years of age, cor pulmonale, the consequence of untreated pulmonary hypertension, is the third most common cardiac disorder. Cardiac diseases produce pulmonary hypertension via volume or pressure overload; although subsequent intimal proliferation of pulmonary resistance vessels adds an obstructive element. Perivascular parenchymal changes along with pulmonary vasoconstriction are the mechanism of pulmonary hypertension in respiratory diseases. Symptoms of pulmonary hypertension include shortness of breath with minimal exertion, fatigue, chest pain, dizzy spells and fainting. Few options are available for the treatment of pulmonary hypertension at this time. Epoprostenol (Flolan), or prostacyclin have been investigated as possible treatments as have inhibitors of platelet aggregation. Inhaled nitric oxide (NO) has also been established as a selective pulmonary vasodilator although problems associated with long-term use of NO inhalation, including its potential toxicity and difficulty in ambulatory inhalation limit its use in the treatment of pulmonary hypertension. Thus other strategies for increasing NO levels or the activity of its signal transduction pathway have been investigated. NO increases cGMP thereby mediating vasodilatation. PDE-III, a second relaxatory molecule, is expressed in the human pulmonary vasulature artery. The activities of PDE-III are increased in models of pulmonary hypertension. This finding along with observations that arterial preparations taken from hypoxic animals respond to PDE-III inhibition (milrinone and SCA40) with a relaxation supports the targeting of this enzyme in human hypertension disease. Most recently, Scottish researchers have investigated the mechanism by which PDE-III activity is increased following chronic hypoxia. PDE-IIIA was found to be over-expressed through a protein kinase A-dependent mechanism. The data further implicates PDE-III in the pathophysiology of pulmonary hypertension, delineate new strategies for targeting these enzymes and support the use of such strategies as therapeutic approaches (Murray et al., 2002).

2. Erectile Dysfunction

Impotence or erectile insufficiency is a widespread disorder that is thought to affect about twelve percent of adult men under age forty-five, about twenty percent of men at age sixty, and about fifty-five percent of men at age seventy-five. Similar to male sexual dysfunction, the prevalence of female sexual dysfunction has been shown to increase with age and be associated with the presence of vascular risk factors and the development of menopause.

There is more than one cause of erectile dysfunction. For example, erectile dysfunction can be psychological, resulting from anxiety or depression, with no apparent somatic or organic impairment. Such erectile dysfunction, which is referred to as “psychogenic,” is responsible for about fifteen to twenty percent of cases of impotence. In other cases, the erectile dysfunction is associated with atherosclerosis of the arteries supplying blood to the penis; such dysfunction is referred to as “arteriogenic” or “atherosclerotic.” About forty to sixty percent of cases of impotence are arteriogenic in origin.

In still other cases, there is leakage from veins in the penis such that sufficient pressure for an erection can be neither obtained nor maintained. This dysfunction is referred to as “venous leakage,” or “abnormal drainage”. This condition is often exacerbated by the presence of some arteriogenic dysfunction whereby the supply of blood to the penis is impaired. In still other cases, the dysfunction is associated with a neuropathy, such as nerve damage arising from, for example, surgery or a pelvic injury, in the nervous system affecting the penis. Such a dysfunction is referred to as “neurogenic” and this accounts for about ten to fifteen percent of cases of impotence.

There is also a high incidence of erectile insufficiency among diabetics, particularly those with insulin-dependent diabetes mellitus. Erectile dysfunction in diabetics is often classified as “diabetogenic,” although the underlying dysfunction is usually neurogenic, but may be arteriogenic or neurogenic and arteriogenic. About half of diabetic males suffer from erectile insufficiency, and about half of the cases of neurogenic impotence are in diabetics.

Additionally, erectile insufficiency is a side effect of certain drugs, such as beta- blockers that are administered to reduce blood pressure in persons suffering from hypertension, or drugs administered to treat depression or anxiety. Excessive alcohol consumption has also been linked to erectile insufficiency. These forms of erectile insufficiency may be regarded as a subset of neurogenic or psychogenic insufficiency.

In humans, penile erection is dependent upon the relaxation of the smooth muscle tone in cells of the corpus cavemosum. This relaxation is dependent on the presence of adequate levels of a cyclic guanosine monophosphate (cyclic GMP) and cyclic adenosine monophosphate (cyclic AMP), which are regulated by phosphodiesterase (PDE) isoenzymes. Cyclic GMP and cyclic AMP are secondary messengers that can be degraded by PDE isoenzymes. The second messenger signal pathway is essential for cavernous smooth muscle relaxation.

A number of methods to treat impotence are available. These treatments include pharmacological treatments, surgery and, in cases of psychogenic dysfunction, psychological counseling is sometimes effective. In the rare cases, where the insufficiency is physical because of venous leakage, surgery can usually be employed to repair the venous lesion and thereby either cure the insufficiency or, if there remains an erectile insufficiency after repair of the venous lesion, render the insufficiency amenable to treatment by pharmacological methods.

As mentioned above, pharmacological methods of treatment are available and shown to be highly effective (U.S. Pat. No. 6,541,487). Treatments for ED include a variety of pharmacologic agents, vacuum devices, and penile prostheses. Among the pharmacologic agents, papaverine, phentolamine, and alprostadil are currently used in practice. These agents are only effective after direct intracavernosal or intraurethral injection, and are associated with side effects such as priapism, fibrosis, penile pain and hematoma at the injection site. Vacuum devices are a noninasive alternative treatment for ED. These devices produce an erection by creating a negative pressure around the shaft of the penis resulting in an increased blood flow into the corpus cavernosum via passive arterial dilation. Although this form of therapy is frequently successful in ED of organic origin, complaints include the lack of spontaneity and the time involved in using a mechanical device, and difficulty and discomfort with ejaculation. A variety of semi-rigid or inflatable penile prostheses have been used with some success, particularly in diabetic men. These devices are generally considered when other treatment options have failed, and are associated with an increased risk of infection and ischemia.

Recently, the selective PDE-V inhibitor, sildenafil (Viagra.RTM) was approved by the FDA as an orally effective medication for the treatment of ED. Sildenafil, 5-[2- ethoxy-5-(4-methylpiperazin-1-ylsulphonyl)phenyl]-1-methyl-3-n-propyl-6,7-dihydro-1H-pyrazolo[4,3-d]pyrimidin-7-one and a number of related analogs and their use as antianginal agents are described in U.S. Pat. Nos. 5,250,534 and 5,346,901. The use of sildenafil and related analogs for treating male erectile dysfunction is described in PCT International Application Publication No. WO 94/28902, published Dec. 22, 1994. In clinical studies, the drug improved sexual function in about 70% of the men who suffer from ED of psychogenic or organic etiology.

PDE-V is likely not the only PDE that is involved in erectile dysfunction. There are seven known types of phosphodiesterase isoenzymes which, if inhibited, affect different functions of the body. Types III PDE's, along with type V, if inhibited, are known to affect the human corpus cavernosum (Stief et al., 1998). For example, the hydrolysis of the second messenger cyclic AMP by PDE-III is known to play an important regulatory role in the relaxation of cavernous smooth muscle of the penis (Kuthe et al., 1999). Sildenafil, unlike enoximone or the enantiomers of enoximone sulfoxide, is a selective PDE-V inhibitor. Sildenafil selectively increases cyclic GMP levels in coronary vascular smooth muscle tissue, but produces no change in cyclic AMP levels. Sildenafil exhibits negligible inhibition of PDE-III, the enzyme targeted by enoximone (Wallis et al., 1999). Thus, both enoximone and the enantiomers of enoximone sulfoxide could be used to treat erectile dysfunction.

3. Other Diseases

PDE-III inhibition has also been indicated or implicated for a variety of other disease states. PDE-III is known to affect platelet aggregation and PDE-III inhibitors may be of use in treating platelet disorders, coagulation and agglutination disorders (Sly et al., 1997). It has been reported that inhibition of PDE-III may be beneficial to alleviate the symptoms of angina (Schlepper et al., 1991). There are a number of reports indicating that PDE-III inhibition could be beneficial in the treatment of renal diseases (Wang et al., 2002; Wagner et al., 1998; Tsuboi et al., 1996; and Takeda et al., 1991). Yamaura et al (2001) have shown that PDE-III inhibition may be useful in the treatment of gastrointestinal disorders. Finally, inhibition of PDE-III has also been indicated for a variety of vascular and circulatory disorders (Ichioka et al., 1998; Shiraishi et al., 1998; and Boldt et al., 1993).

### III. Enoximone

Enoximone (1,3-Dihydro-4-methyl-5-[4-(methylthio)benzoyl]-2H-imidazol-2-one) is a small organic molecule that exhibits highly selective inhibition of type-III phosphodiesterase, or PDE-III, an enzyme that is present in the heart and plays an important regulatory role in cardiac function. PDE-III inhibitors block the action of this enzyme, increasing the force of contraction of the heart, thereby increasing cardiac output. Compounds that increase the force of contraction of the heart, like enoximone, are referred to as positive inotropes. Enoximone also causes vasodilation, an increase in the diameter of blood vessels, through its effects on smooth muscle cells that surround blood vessels, which results in lower pressure against which the heart must pump. Positive inotropy and vasodilation can both be therapeutically useful in the treatment of heart failure. Enoximone is described in detail in U.S. Pat. No. 4,505,635, which is hereby incorporated by reference.

Patients with advanced chronic heart failure can benefit greatly from the chronic use of an oral inotropic agent that would provide the desired symptomatic relief to the patients and reduce the frequency of hospitalizations by delaying additional episodes of acute decompensated heart failure. An oral product with these characteristics could also wean patients with severe heart failure who are currently dependent on intravenous inotropic therapy from those agents and allow them the opportunity to leave the hospital and return to a more normal daily life. Such an agent would decrease the overall costs associated with the treatment of heart failure. While enoximone represents such an agent, the enantiomers of enoximone sulfoxide represent another active and new PDE-III inhibitor that could be used to treat not only chronic heart failure, but any disease state in which inhibition of PDE-III is indicated.

### A. Sulfoxide Enantiomers

As stated above, Enoximone belongs to the imidazole class of compounds that possess positive inotropic and vasodilatory activities. These pharmacologic effects are caused by selective inhibition of a PDE-III in the heart and in the smooth muscle of blood vessels. Results obtained in intact animals show a dose-dependent increase in cardiac contractile force and a reduction in peripheral arterial resistance with only a slight increase in heart rate. Following acute administration of enoximone to patients suffering from cardiac failure an almost linear increase of cardiac index with increasing doses was found. Enoximone is eliminated from the body both unchanged and after biotransformation. Sulfoxide formation is the main metabolic transformation in man. This metabolite is excreted in the urine. Enoximone sulfoxide also possesses cardiotonic activity. Reconversion of enoximone sulfoxide to enoximone was shown to occur in the liver and, to some extent, also in the kidney. Bioavailability of enoximone after a single oral dose of 3 mg/kg is about 55%, but may be higher following chronic therapy. This is probably due to saturation of the first-pass metabolism. A mean clearance of about 10 ml/min/kg and a mean half-life of 6 h were determined in patients with cardiac failure. These values are different from those measured in normal volunteers, indicating a reduced clearance of enoximone in these patients. In patients with renal failure enoximone sulfoxide accumulates in plasma. The elimination of enoximone is also reduced (Jahnchen & Trenk, 1991).

Previously various labs studied the absorption and disposition kinetics of enoximone and enoximone sulfoxide in humans, after both single oral doses of enoximone and at steady-state after short-term chronic oral therapy. (Ruder et al., 1991) The plasma levels of enoximone sulfoxide were seen to be greater than those of enoximone at all sampling times. The peak enoximone sulfoxide plasma concentrations ranged from 3.5 to 17.3 times the peak enoximone plasma levels for individual patients. (Ruder et al., 1991) The average steady-state plasma concentrations for enoximone were 115+/−40 ng/mL and 190+/−78 ng/mL for 50 mg every 8 hours and 100 mg every 8 hours dosage regimens, respectively (Ruder et al. 1991). The absorption and disposition kinetics of enoximone were found to be significantly variable. The relationship between dose administered and steady-state plasma levels as well as the relationship between the observed and predicted steady-state plasma levels was also studied. It was found that there was a linear relationship between the dose that was administered and the accrued plasma levels, as well as a good correlation between the predicted and observed steady-state levels. Although the data confirmed previous reports that the sulfide metabolite of enoximone accumulated extensively in the plasma during oral therapy, reaching levels much higher than those of enoximone, the early research data did not support the use of the sulfoxide as a drug. (Morita et al., 1995).

Enoximone sulfoxide is chiral. It has now been shown by the inventors that the (S)-(−)-enantiomer of enoximone sulfoxide is more active than the (R)-(+)-enantiomer. As such, a purified version of the (S)-(−)-enantiomer of enoximone sulfoxide is presented in this invention as a therapeutic compound for use in the treatment of a variety of diseases for which inhibition of PDE-III may be beneficial.

### B. Synthesis of Enoximone

Enoximone can be obtained in a variety of ways (see U.S. Pat. No. 4,405,635; Schnettler et al., J. Med. Chem., 25: 1477-1481, 1982).

### C. Synthesis of 1,3-Dihydro-4-methyl-5-[(4-methylsulfinyl)-benzoyl]-2H- imidazol-2-one hydrate (Enoximone Sulfoxide)

A mixture of 496 grams (2.0 moles) of enoximone and 34.7 liters of acetic acid was charged to a 72 liter flask fitted with a stirrer, thermometer and dropping funnel. The resulting mixture was stirred while adding 227 grams (2.0 moles) of 30% hydrogen peroxide in a slow stream. Stirring at ambient temperature was maintained for 72 hours. The pot temperature ranged from a low of 17° C. to a high of 25° C. during the 72 hours.

A peroxide test with starch-iodine paper was negative after 72 hours. Acetic acid was evaporated in vacuo at 40° C. The solid residue obtained was slurried in 2 liters of water. Crude product was filtered, washed well with water, and then air dried at ambient temperature to yield 510 grams, dec. 273-275° C.

A total of 995 grams of crude product, prepared as described above, was dissolved in 10 liters of dimethylformamide at 146° C. The hot solution was gravity filtered to remove the insolubles present, then stored at room temperature for 6.5 hours (pot temperature at the end was 30° C.). Product was filtered, washed with 3×1 liter of cold dimethylformamide, and then dried at ambient temperature to yield 790 grams, dec. 274-275° C.

A total of 789 grams (2.98 moles) of the above semi-purified product was charged into a 12 liter round bottom flask along with 5.544 liters of water. The resulting suspension was vigorously stirred while adding a solution of 118.8 grams (2.97 moles) of sodium hydroxide in 1.544 liters of water over a period of 30 minutes. Once solution was obtained, 78.7 grams of Nuchar was added and the mixture was stirred for 15 minutes at room temperature. Charcoal was removed by filtration through celite, using 1.44 liters of water for rinse. The resulting filtrate was stirred while adding 0.96 liters of 10% hydrochloric acid over a period of 30 minutes. After stirring an additional 30 minutes, product was filtered, washed 3×2 liters of ice water, then air dried at ambient temperature to yield 755 grams, dec. 276-277° C., 63.5% yield.

Elemental Analysis: Calc'd. for C₁₂H₁₂N₂O₃S.H₂O: C, 51.05; H, 5.00; N, 9.93; S, 11.36. Found: C, 50.90; H, 5.01; N, 9.92; S, 11.35.

### D. Synthesis of the (R)-(+)- and (S)-(−)-Enantiomers of 1,3-Dihydro-4-methyl-5-[(4-methylsulfinyl)-benzoyl]-2H-imidazol-2-one hydrate

(Enoximone Sulfoxide)

1. Method A

The (R)-(+)- and (S)-(−)-enantiomers of enoximone sulfoxide may be prepared from racemic enoximone sulfoxide by preparative chromatographic separation using a chiral solid phase. High-performance liquid chromatography (HPLC) is the most common technique used for such a separation. High pressure, medium pressure, low pressure and atmosphere pressure liquid chromatography can be used for such a separation. Many liquid phases and chiral solid phases are available for this type of application. For a review of methods see Francotte (2001); and Anderson and Allenmark (2002), hereinafter incorporated by reference. For a review of semipreparative applications see Inotsume and Nakano (2002); and Boatto et al. (2003); and related examples can be found in Dolle et al. (1997); and Alajarin et al. (1995), all of which are hereinafter incorporated by reference.

In other embodiments of Method A the racemate may first be derivatized with an achiral or chiral derivatizing agent to enhance the resolution and separation efficiency; the solid phase may be prepared as an imprinted polymer from one or the other of the enantiomers to be separated; in some cases separations may be achieved using an achiral solid phase with chiral additives in the liquid phase; in some. cases separations may be achieved using an achiral solid phase and a racemate derivatized with a chiral reagent (for a review see Toyo'oka, 2002).

2. Method B

The racemic enoximone sulfoxide may first be reacted with a chiral derivatizing agent(s) to yield a mixture of diastereomers. These diastereomers may then be separated by one skilled in the art using standard techniques such as crystallization or chromatography. Following separation and isolation of the individual diastereomers the chiral derivatizing group previously added is removed using methods known by one skilled in the art and the individual pure enantiomers are obtained, further purified if necessary, and characterized (March, 1992).

3. Method C

The desired enantiomer of enoximone sulfoxide may be prepared by one skilled in the art through the application of chiral or asymmetric synthesis. In this method a chiral and optically active staring material or building block added during the synthesis dictates the enantiomer synthesized. In another embodiment of Method C a chiral reagent, not incorporated into the final compound, is used during the synthesis to direct selective formation of chirality in the compound with formation of a single enantiomer (for general reviews see Burke and Henderson, 2002; Hillier and Reider, 2002; and Iida and Mase, 2002).

In another embodiment of Method C one skilled in the art may be able to apply bioprocesses to the asymmetric synthesis of the desired enantiomers (for a review see Patel, 2001; and Huisman and Gray, 2002).

In another embodiment of Method C one skilled in the art may be able to use deracemization at some point during a synthesis of the desired enantiomers. Deracemization processes may be afforded by either bioprocess or non-bioprocess techniques (March, 1992). In another embodiment of Method C one skilled in the art may be able to use kinetic resolution to achieve an asymmetric synthesis or separation of the desired enantiomers (March, 1992).

E. Optical Rotation for (S)-(−) Enantiomer
[α]_{D} at 25 C=−53.3° (c=0.5, DMSO)

### IV. Methods of Treatment

A. Exemplary Therapeutic Regimens for Heart Failure and Hypertrophy

Heart failure of some forms may be curable, and these are dealt with by treating the primary disease, such as anemia or thyrotoxicosis. Also curable are forms caused by anatomical problems, such as a heart valve defect. These defects can be surgically corrected. However, for the most common forms of heart failure—those due to damaged heart muscle—no known cure exists. Treating the symptoms of these diseases helps, and some treatments of the disease have been successful. The treatments attempt to improve patients' quality of life and length of survival through lifestyle change and drug therapy. Patients can minimize the effects of heart failure by controlling the risk factors for heart disease, but even with lifestyle changes, most heart failure patients must take medication, many of whom receive two or more drugs.

The pharmacological treatment of heart failure may serve as an example of how PDE-III inhibitors could be used to treat any of a variety of diseases. Several types of drugs have proven helpful in the treatment of heart failure, but none in and of themselves have proven to be universally effective or able to fully control the disease. Diuretics can help reduce the amount of fluid in the body and are useful for patients with fluid retention and hypertension; and digitalis can be used to increase the force of the heart's contractions, helping to improve circulation. Results of recent studies have placed more emphasis on the use of ACE inhibitors (Manoria and Manoria, 2003). Several large studies have indicated that ACE inhibitors improve survival among heart failure patients and may slow, or perhaps even prevent, the loss of heart pumping activity (for a review see De Feo et al., 2003; DiBianco, 2003). Patients who cannot take ACE inhibitors may get a nitrate and/or a drug called hydralazine, each of which helps relax tension in blood vessels to improve blood flow (Ahmed, 2003). But, as mentioned above, these drugs are not curative and there is a strong need for better pharmaceuticals.

To date, no alternative treatments (surgical or otherwise) have been shown to cure heart failure, but like the aforementioned drug treatments, some alternative therapies can at least improve quality of life and extend life for those suffering this disease.

As with heart failure, there are no known cures to hypertrophy. Current medical management of cardiac hypertrophy, in the setting of a cardiovascular disorder includes the use of at least two types of drugs: inhibitors of the rennin-angiotensoin system, and β-adrenergic blocking agents (Bristow, 1999). Therapeutic agents to treat pathologic hypertrophy in the setting of heart failure include angiotensin II converting enzyme (ACE) inhibitors and β-adrenergic receptor blocking agents (Eichhom and Bristow, 1996). Other pharmaceutical agents that have been disclosed for treatment of cardiac hypertrophy include angiotensin II receptor antagonists (U.S. Pat. No. 5,604,251) and neuropeptide Y antagonists (WO 98/33791).

Non-pharmacological treatment is primarily used as an adjunct to pharmacological treatment. One means of non-pharmacological treatment involves reducing the sodium in the diet. In addition, non-pharmacological treatment also entails the elimination of certain precipitating drugs, including negative inotropic agents (e.g., certain calcium channel blockers and antiarrhythmic drugs like disopyramide), cardiotoxins (e.g., amphetamines), and plasma volume expanders (e.g., nonsteroidal anti- inflammatory agents and glucocorticoids).

As can be seen from the discussion above, there is a great need for a successful treatment approach to heart failure and hypertrophy. In one embodiment of the present invention, methods for the treatment of cardiac hypertrophy or heart failure utilizing formulations comprising a purified enoximone sulfoxide (S)-(−)-enantiomer are disclosed. For the purposes of the present application, treatment comprises reducing one or more of the symptoms of any disease state where inhibition of PDE-III would be considered beneficial, for example in heart failure or cardiac hypertrophy. Symptoms for heart disease might be reduced exercise capacity, reduced blood ejection volume, increased left ventricular end diastolic pressure, increased pulmonary capillary wedge pressure, reduced cardiac output, cardiac index, increased pulmonary artery pressures, increased left ventricular end systolic and diastolic dimensions, and increased left ventricular wall stress, wall tension and wall thickness-same for right ventricle. In addition, use of inhibitors of PDE-III such as the purified enoximone sulfoxide enantiomers may prevent cardiac hypertrophy and its associated symptoms from arising.

B. Combined Therapy

In another embodiment, it is envisioned to use the enoximone sulfoxide (−)- enantiomer in combination with other therapeutic modalities. Thus, in addition to the therapies described above, one may also provide to the patient more “standard” pharmaceutical cardiac therapies. Examples of other therapies include, without limitation, so-called “beta blockers,” anti-hypertensives, cardiotonics, anti-thrombotics, vasodilators, hormone antagonists, other inotropes, diuretics, endothelin antagonists, calcium channel blockers, phosphodiesterase inhibitors, ACE inhibitors, angiotensin type 2 antagonists and cytokine blockers/inhibitors, and HDAC inhibitors.

Combinations may be achieved by contacting cardiac cells with a single composition or pharmacological formulation that includes both agents, or by contacting the cell with two distinct compositions or formulations, at the same time, wherein one composition includes the expression construct and the other includes the agent. Alternatively, the therapy using the enoximone sulfoxide (S)-(−)-enantiomer may precede or follow administration of the other agent(s) by intervals ranging from minutes to weeks. In embodiments where the other agent and the enoximone sulfoxide enantiomer are applied separately to the cell, one would generally ensure that a significant period of time did not expire between the time of each delivery, such that the agent and the enoximone sulfoxide enantiomer would still be able to exert an advantageously combined effect on the cell. in such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the enoximone sulfoxide enantiomer or the other agent will be desired. In this regard, various combinations may be employed. By way of illustration, where the enoximone sulfoxide enantiomer is “A” and the other agent is “B,” the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated.

C. Adjunct Therapeutic Agents for Combination Therapy

Pharmacological therapeutic agents and methods of administration, dosages, etc., are well known to those of skill in the art (see for example, the “Physicians Desk Reference,” Goodman and Gilman's “The Pharmacological Basis of Therapeutics, Tenth Edition” “Remington's Pharmaceutical Sciences,” and “The Merck Index, Thirteenth Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

1. Antihyperlipoproteinemics

In certain embodiments, administration of an agent that lowers the concentration of one of more blood lipids and/or lipoproteins, known herein as an “antihyperlipoproteinemic,” may be combined with a cardiovascular therapy according to the present invention, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.

a. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

b. Resins/Bile Acid Sequesterants

Non-limiting examples of resins/bile acid sequesterants include cholestyramine (cholybar, questran), colestipol (colestid) and polidexide.

c. HMG CoA Reductase Inhibitors

Non-limiting examples of HMG CoA reductase inhibitors include lovastatin (mevacor), pravastatin (pravochol) or simvastatin (zocor).

d. Nicotinic Acid Derivatives

Non-limiting examples of nicotinic acid derivatives include nicotinate, acepimox, niceritrol, nicoclonate, nicomol and oxiniacic acid.

e. Thryroid Hormones and Analogs

Non-limiting examples of thyroid hormones and analogs thereof include etoroxate, thyropropic acid and thyroxine.

f. Miscellaneous Antihyperlipoproteinemics

Non-limiting examples of miscellaneous antihyperlipoproteinemics include acifran, azacosterol, benfluorex, b-benzalbutyramide, camitine, chondroitin sulfate, clomestrone, detaxtran, dextran sulfate sodium, 5,8,11,14,17-eicosapentaenoic acid, eritadenine, furazabol, meglutol, melinamide, mytatrienediol, ornithine, g-oryzanol, pantethine, pentaerythritol tetraacetate, a-phenylbutyramide, pirozadil, probucol (lorelco), b-sitosterol, sultosilic acid-piperazine salt, tiadenol, triparanol and xenbucin.

2. Antiarteriosclerotics

Non-limiting examples of an anti arteriosclerotic include pyridinol carbamate.

3. Antithrombotic/Fibrinolytic Agents

In certain embodiments, administration of an agent that aids in the removal or prevention of blood clots may be combined with administration of a modulator, particularly in treatment of athersclerosis and vasculature (e.g., arterial) blockages. Non- limiting examples of antithrombotic and/or fibrinolytic agents include anticoagulants, anticoagulant antagonists, antiplatelet agents, thrombo lyric agents, thrombolytic agent antagonists or combinations thereof.

In certain aspects, antithrombotic agents that can be administered orally, such as, for example, aspirin and wafarin (coumadin), are preferred.

a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

b. Antiplatelet Agents

Non-limiting examples of antiplatelet agents include aspirin, a dextran, dipyridamole (persantin), heparin, sulfinpyranone (anturane) and ticlopidine (ticlid).

c. Thrombolytic Agents

Non-limiting examples of thrombolytic agents include tissue plasminogen activator (activase), plasmin, pro-urokinase, urokinase (abbokinase) streptokinase (streptase), anistreplase/APSAC (eminase).

4. Blood Coagulants

In certain embodiments wherein a patient is suffering from a hemhorrage or an increased likelyhood of hemhorraging, an agent that may enhance blood coagulation may be used. Non-limiting examples of a blood coagulation promoting agent include thrombolytic agent antagonists and anticoagulant antagonists.

a. Anticoagulant Antagonists

Non-limiting examples of anticoagulant antagonists include protamine and vitamine KI.

b. Thrombolytic Agent Antagonists and Antithrombotics

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

5. Antiarrhythmic Agents

Non-limiting examples of antiarrhythmic agents include Class I antiarrhythmic agents (sodium channel blockers), Class II antiarrhythmic agents (beta-adrenergic blockers), Class II antiarrhythmic agents (repolarization prolonging drugs), Class IV antiarrhythmic agents (calcium channel blockers) and miscellaneous antiarrhythmic agents.

a. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocalne), tocainide (tonocard) and mexiletine (mexitil). Non-limiting examples of Class IC antiarrhythmic agents include encainide (enkaid) and flecainide (tambocor).

b. Beta Blockers

Non-limiting examples of a beta blocker, otherwise known as a b-adrenergic blocker, a b-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

c. Repolarization Prolonging Agents

Non-limiting examples of an agent that prolong repolarization, also known as a Class III antiarrhythmic agent, include amiodarone (cordarone) and sotalol (betapace).

d. Calcium Channel Blockers/Antagonist

Non-limiting examples of a calcium channel blocker, otherwise known as a Class IV antiarrhythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexiline. In certain embodiments a calcium channel blocker comprises a long-acting dihydropyridine (amlodipine) calcium antagonist.

e. Miscellaneous Antiarrhythmic Agents

Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyramide, hydroquinidine, indecainide, ipatropium bromide, lidocaine, lorajmine, lorcainide, meobentine, moricizine, pirmenol, prajmaline, propafenone, pyrinoline, quinidine polygalacturonate, quinidine sulfate and viquidil.

6. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.

a. Alpha Blockers

Non-limiting examples of an alpha blocker, also known as an a-adrenergic blocker or an a-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

b. Alpha/Beta Blockers

In certain embodiments, an antihypertensive agent is both an alpha and beta adrenergic antagonist. Non-limiting examples of an alpha/beta blocker comprise labetalol (normodyne, trandate).

c. Anti-Angiotension II Agents

Non-limiting examples of anti-angiotension II agents include include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan.

d. Sympatholytics

Non-limiting examples of a sympatholytic include a centrally acting sympatholytic or a peripherially acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a B-adrenergic blocker include acenitolol (sectral), atenolol (tenornin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin).

e. Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(b-diethylaminoethyl ether), hexobendine, itramin tosylate, khellin, lidoflanine, mannitol hexanitrane, medibazine, nicorglycerin, pentaerythritol tetranitrate, pentrinitrol, perhexiline, pimethylline, trapidil, tricromyl, trimetazidine, troInitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

f. Miscellaneous Antihypertensives

Non-limiting examples of miscellaneous antihypertensives include ajmaline, gama-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative.

Arylethanolamine Derivatives. Non-limiting examples of arylethanolamine derivatives include amosulalol, bufuralol, dilevalol, labetalol, pronethalol, sotalol and sulfinalol.

Benzothiadiazine Derivatives. Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-carboxyalkyl(peptide/lactam) Derivatives. Non-limiting examples of N- carboxyalkyl(peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives. Non-limiting examples of dihydropyridine derivatives include amlodipine, felodipine, isradipine, nicardipine, nifedipine, nilvadipine, nisoldipine and nitrendipine.

Guanidine Derivatives. Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines. Non-limiting examples of hydrazines/phthalazines include budralazine, cadralazine, dihydralazine, endralazine, hydracarbazine, hydralazine, pheniprazine, pildralazine and todralazine.

Imidazole Derivatives. Non-limiting examples of imidazole derivatives include clonidine, lofexidine, phentolamine, tiamenidine and tolonidine.

Quanternary Ammonium Compounds. Non-limiting examples of quanternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives. Non-limiting examples of reserpine derivatives include bietaserpine, deserpidine, rescinnamine, reserpine and syrosingopine.

Suflonamide Derivatives. Non-limiting examples of sulfonamide derivatives include ambuside, clopamide, furosemide, indapamide, quinethazone, tripamide and xipamide.

7. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

8. Treatment Agents for Congestive Heart Failure

Non-limiting examples of agents for the treatment of congestive heart failure include anti-angiotension II agents, afterload-preload reduction treatment, diuretics and inotropic agents.

a. Afterload-Preload Reduction

In certain embodiments, an animal patient that can not tolerate an angiotension antagonist may be treated with a combination therapy. Such therapy may combine adminstration of hydralazine (apresoline) and isosorbide dinitrate (isordil, sorbitrate).

b. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetamide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretamide, quinethazone, torasemide, tripamide, xipamide), a uracil (e.g., aminometradine, amisometradine), a potassium sparing antagonist (e.g., amiloride, triamterene)or a miscellaneous diuretic such as aminozine, arbutin, chlorazanil, ethacrynic acid, etozolin, hydracarbazine, isosorbide, mannitol, metochalcone, muzolimine, perhexiline, ticmafen and urea.

c. Other Inotropic Agents

Non-limiting examples of a positive inotropic agent, also known as a cardiotonic, include acefylline, an acetyldigitoxin, 2-amino-4-picoline, amrinone, benfulrodil hemisuccinate, bucladesine, cerberosine, camphotamide, convallatoxin, cymarin, denopamine, deslanoside, digitalin, digitalis, digitoxin, digoxin, dobutamine, dopamine, dopexamine, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β- adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include amrinone (inocor).

d. Antianginal Agents

Antianginal agents may comprise organonitrates, calcium channel blockers, beta blockers and combinations thereof. Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

9. Surgical Therapeutic Agents

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

D. Formulations and Routes of Administration for Other Agents

It will be understood that in the discussion of formulations and methods of treatment, references to any compounds are meant to also include the pharmaceutically acceptable salts, as well as pharmaceutical compositions. It is further understood that treatment methods disclosed may be applied to any of the disease states mentioned in the application. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector or cells, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

In specific embodiments of the invention the pharmaceutical formulation will be formulated for delivery via rapid release, other embodiments contemplated include but are not limited to timed release, delayed release, and sustained release. Formulations can be an oral suspension in either the solid or liquid form. In further embodiments, it is contemplated that the formulation can be prepared for delivery via parenteral delivery, by dilution into a drip bag, or used as a suppository, or be formulated for subcutaneous, intravenous, intramuscular, intraperitoneal, sublingual, transdermal, or nasopharyngeal delivery.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release (hereinafter incorporated by reference).

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain an active material in admixture with excipients suitable for the manufacture of aqueous suspensions. Such excipients are suspending agents, for example sodium carboxymethylcellulose, methylcellulose, hydroxy- propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. Pharmaceutical compositions may be in the form of a sterile injectable aqueous or oleagenous suspension. Suspensions may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds may also be administered in the form of suppositories for rectal administration of the drug. These compositions can be prepared by mixing a therapeutic agent with a suitable non-irritating excipient which is solid at ordinary temperatures, but liquid at the rectal temperature and will therefore melt in the rectum to release the drug. Such materials are cocoa butter and polyethylene glycols.

For topical use, creams, ointments, jellies, gels, epidermal solutions or suspensions, etc., containing a therapeutic compound are employed. For purposes of this application, topical application shall include mouthwashes and gargles.

Formulations may also be administered as nanoparticles, liposomes, granules, inhalants, nasal solutions, or intravenous admixtures

The previously mentioned formulations are all contemplated for treating patients suffering from heart failure or hypertrophy. The amount of active ingredient in any formulation may vary to produce a dosage form that will depend on the particular treatment and mode of administration. It is further understood that specific dosing for a patient will depend upon a variety of factors including age, body weight, general health, sex, diet, time of administration, route of administration, rate of excretion, drug combination and the severity of the particular disease undergoing therapy.

### V. Definitions

As used herein, the term “heart failure” is broadly used to mean any condition that reduces the ability of the heart to pump blood. As a result, congestion and edema develop in the tissues. Most frequently, heart failure is caused by decreased contractility of the myocardium, resulting from reduced coronary blood flow; however, many other factors may result in heart failure, including damage to the heart valves, vitamin deficiency, and primary cardiac muscle disease. Though the precise physiological mechanisms of heart failure are not entirely understood, heart failure is generally believed to involve disorders in several cardiac autonomic properties, including sympathetic, parasympathetic, and baroreceptor responses. The phrase “manifestations of heart failure” is used broadly to encompass all of the sequelae associated with heart failure, such as shortness of breath, pitting edema, an enlarged tender liver, engorged neck veins, pulmonary rales and the like including laboratory findings associated with heart failure.

The term “treatment” or grammatical equivalents encompasses the improvement and/or reversal of the symptoms of heart failure (i.e., the ability of the heart to pump blood). “Improvement in the physiologic function” of the heart may be assessed using any of the measurements described herein (e.g., measurement of ejection fraction, fractional shortening, left ventricular internal dimension, heart rate, etc.), as well as any effect upon the animal's survival. In use of animal models, the response of treated transgenic animals and untreated transgenic animals is compared using any of the assays described herein (in addition, treated and untreated non-transgenic animals may be included as controls). A compound which causes an improvement in any parameter associated with heart failure used in the screening methods of the instant invention may thereby be identified as a therapeutic compound.

As used herein, the term “cardiac hypertrophy” refers to the process in which adult cardiac myocytes respond to stress through hypertrophic growth. Such growth is characterized by cell size increases without cell division, assembling of additional sarcomeres within the cell to maximize force generation, and an activation of a fetal cardiac gene program. Cardiac hypertrophy is often associated with increased risk of morbidity and mortality, and thus studies aimed at understanding the molecular mechanisms of cardiac hypertrophy could have a significant impact on human health.

As used herein, the term “modulate” refers to a change or an alteration in a biological activity. Modulation may be an increase or a decrease in protein activity, by action of an agonist (which is an agent capable of stimulating an activity) or an antagonist (which is an agent capable of inhibiting an activity), a change in kinase activity, a change in binding characteristics, or any other change in the biological, functional, or immunological properties associated with the-activity of a protein or other structure of interest. The term “modulator” refers to any molecule or compound which is capable of changing or altering biological activity as described above.

### VI. EXAMPLES

A. Materials and Methods

Explanted human hearts were obtained from transplant recipients (n=7) and from nonfailing donor hearts (n=1). After excision, hearts were placed immediately into ice cold Tyrode's solution and aerated with 95% O₂ and 5% CO₂. Trabeculae between 1-3 mm in width and 7-10 mm in length were excised from the right ventricular free wall. Subsequently trabeculae were mounted in a multi-chamber muscle bath in oxygenated Tyrode's solution at 37° C. The trabeculae were placed under 1 gram of resting tension and a field stimulation of 1.0 Hz. After a 2 hour incubation period, trabeculae were exposed to increasing concentrations of Enoximone sulfoxide enantiomers (10⁻⁷ to 10⁻⁴), isoproterenol (10⁻⁹ to 10⁻⁴) and vehicle control (DMAC and NaOH). Peak contractile forces were recorded at 10 minute intervals for a total of 1½ hours. Following experimentation, trabeculae were frozen in liquid N2.

B. Data

The tension study normalized the peak contraction value relative to baseline measurements generated by each subject in the absence of added agonists at T=0. Isoproterenol was used as a positive control and showed a marked increase in contraction tension when titrated (>1000 mg of tension). Net contractile force was then determined by subtracting the T=0 value from each subsequent measurement. Enoximone showed a peak contractile force equivalent to 575 mg of tension. Sulfoxide Metabolite 27996, the (R)-(+)-enantiomer, was found to have less increased contractility, 175 mg, when compared to the (S)-(−)-enantiomer isomer, which had a significant increase in contractility, 550 mg, nearly the same tension as measured for Enoximone.

C. PDE3 Inhibition of Enantiomer

Enoximone Sulfoxide enantiomer was tested for its ability to inhibit the action of PDE-III isolated from human platelets. The substrate was [³H]cAMP+cAMP, which is converted by PDE-III to [³H]Adenosine and then [³H]Adenosine can be quantified. Assays were done in triplicate at three different concentrations over log scale, allowing semi-quantitative measurement of IC₅₀ The IC₅₀ for the (S)-(−)-enantiomer of enoximone sulfoxide was determined to be 147 μM by this assay.

### VII. References

The following references, to the extent that they provide exemplary procedural or other details supplementary to those set forth herein, are specifically incorporated herein by reference.
U.S. Pat. No. 4,166,452
U.S. Pat. No. 4,256,108
U.S. Pat. No. 4,265,874
U.S. Pat. No. 4,405,635
U.S. Pat. No. 4,505,635
U.S. Pat. No. 5,250,534
U.S. Pat. No. 5,346,901
U.S. Pat. No. 5,604,251
U.S. Pat. No. 5,998,458
U.S. Pat. No. 6,254,885
U.S. Pat. No. 6,555,135
U.S. Pat. No. 6,596,308
U.S. Pat. No. 6,623,760
U.S. Pat. No. 6,541,487
U.S. Pat. No. 6,645,466
Ahmed, Am. Geriatr. Soc., 51(1):123-126, 2003.
Alajarin et al., J. Medicinal Chem., 38:2830, 1995.
Anderson and Allenmark, J. Biochem. Biophysical Methods, 54:11, 2002.
Boatto et al., Chirality, 15:494, 2003.
Boldt et al. Br. J. Clin. Pharmacol., 36:309-314, 1993.
Bristow, Z. Kardiol., 83(2):15-19, 1994.
Bristow, Cardiology, 92:3-6, 1999.
Burke and Henderson, Brit. J. Anaesthesia, 88:563, 2002.
Conte et al., J. Heart Lung Transplant., 17:679-685, 1998.
De Feo et al., Ital. Heart J., 4:511-513, 2003.
DiBianco, Am. J. Med., 115:480-488, 2003.
Doherty, “Oral, Transdermal and Transurethral Therapies for Erectile Dysfunction” in Male Infertility and Dysfunction, Hellstrom, ed., Chapter 34 (New York, N.Y.: Springer-Verlag, 1997.
Dolle et al., Bioorganic Med. Chem., 5:749, 1997.
Dumcius et al., Medicina, 39:815-822, 2003.
Durand et al., Ann. Med., 27:311-317, 1995.
Eichhom and Bristow, Circulation, 94:2285-2296, 1996.
Francotte, J. Chromatography A, 906:379, 2001.
Goodman and Gilman's The Pharmacological Basis Of Therapeutics, Hardman et al. (Eds.), 10^{th} ed., 32:853-860; 35:891-893, 2001.
Handbook of Pharmaceutical Excipients, 3d Ed., Kibbe (Ed.), 2000.
Handbook of Industrial Surfactants, 3d Ed. Vol. 1 & 2, Ash (Ed.), 2000.
Hillier and Reider, Drug Discovery Today, 7:303, 2002.
Huisman and Gray, Current Opinion in Biotechnology, 13:352, 2002.
Iida and Mase, Current Opinion in Drug Discovery and Development, 5:834, 2002.
Ichioka et al, Surg Res. 75(1):42-8, 1998.
Inotsume and Nakano, J. Biochem. Biophysical Methods, 54:255, 2002.
Jahnchen & Trenk, Z. Kardiol. (German), 80 Supp:21-26, 1991.
Komas et al., Phosphodiesterase Inhibitors, Eds. Schudt et al., Ch. 6 (San Diego, Calif.: Academic Press, 1996).
Kuthe et al., Chem. Biol. Interact., 119-120:593-8, 1999.
Levy et al., N. Engl. J. Med., 322:1561-1566, 1990.
Manoria and Manoria, J. Indian. Med. Assoc., 101(5):311-312, 2003.
March, Advanced Organic Chemistry, 4^{th} ed., Chapter 4, page 94, 1992.
Merck Index, Thirteenth Edition
Morita et al., J. Pharm. Sci., 84:152=157, 1995.
Murray et al., Br. J. Pharmacol., 137:1187-1194, 2002.
Patel, Current Opinion in Biotech., 12:587, 2001.
PCT Appl. WO 98/33791
PCT Appl. WO 96/16644
PCT Appl. WO 94/28902
Physicians Desk Reference.
Ruder et al., J. Clin. Pharmacol., 21:702-708, 1991.
Schlepper et al., Z Kardiol. 80 Supp 4:75-83, 1991.
Schnettler et al., J. Med. Chem., 25: 1477-1481, 1982.
Schudt et al., Eur. Respir. J, 8:1179-1183, 1995.
Shiraishi et al., Br. J. Pharacol., 123:869-878, 1998.
Sly et al., Shock, 8:115-118, 1997.
Stief et al, J. Urol., 159(4):1390-3, 1998.
Takeda et al., Endocrinology, 129:287-294, 1991.
Toyo'oka, J. Biochemical and Biophysical Methods, 54:25, 2002.
Tsuboi et al., J. Clin. Invest., 98:262-270, 1996.
Wagner et al., Kidney Int. Suppl., 67:S78-83, 1998.
Wallis et al., Am. J. Cardiol., 83(5A):3C-12C, 1999.
Wang et al., Am J Physiol Renal Physiol., 283:F1313-1325, 2002.
Yamaura et al., Acta Anaesthesiol Scand. 45(4):427-34, 2001.
Young et al, Handbook of Applied Therapeutics, 7.1-7.12 and 9.1-9.10, 1989.

What is claimed is:

1. A composition comprising an optically active compound having the Formula I: 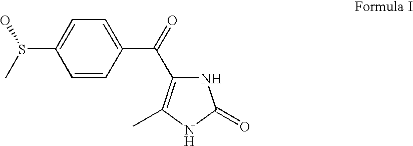
and wherein the composition is the (S)-(−)-form and is substantially free of the (R)-(+)- form; and pharmaceutically acceptable salts thereof.

2. The composition of claim 1, wherein the (S)-(−) enantiomer of said compound is greater than 70% pure, greater than 75% pure, greater than 80% pure, greater than 85% pure, greater than 90% pure, greater than 95% pure, greater than 96% pure, greater than 97% pure, greater than 98% pure, or greater than 99% pure.

3. A pharmaceutical formulation comprising the compound of formula I, and pharmaceutically acceptable salts thereof, wherein the compound is the (S)-(−)-form and is substantially free of the (R)-(+)-form.

4. The formulation of claim 3, formulated for delivery via rapid release, timed release, delayed release, sustained release, oral suspension, parenteral delivery, suppository, subcutaneous, intravenous, intramuscular, intraperitoneal, sublingual, transdermal or nasopharyngeal routes.

5. The formulation of claim 4, wherein the compound is in a solid form.

6. The formulation of claim 4, wherein the compound is in a liquid form.

7. The formulation of claim 4, wherein the compound is formulated as an uncoated tablet, as a coated tablet, a capsule, a powder, a troche, a granule, a liposome, a suppository, a solution, a colloid, an ointment, a cream, a vapor, a spray, a nanoparticle, an inhalant, a nasal solution, an intravenous admixture, an epidermal solution, a buccal tablet, a syrup, a cream, a lotion, a gel, an emulsion, or an elixer.

8. The formulation of claim 7, further comprising one or more of a tablet binder, a filler, a preservative, a tablet disintegrant, a flow regulator, a plasticizer, a wetting agent, a dispersant, an emulsifier, a solvent, a release-slowing agent, an antioxidant, or a propellant gas.

9. A method for treating a disease state in a patient where PDE-III inhibition is beneficial, comprising administration of a pharmaceutical formulation comprising the compound of formula I, and pharmaceutically acceptable salts thereof, wherein the compound is the (S)-(−)-form and is substantially free of the (R)-(+)-form.

10. The method of claim 9, wherein said disease state is selected from one or more of acute heart failure, chronic heart failure, hemodynamic failure, chronic heart disease, or cardiac hypertrophy.

11. The method of claim 9, wherein said disease state is selected from one or more of platelet disorder, renal disease, renal failure, pulmonary hypertension, PAH, stable angina, unstable angina, erectile dysfunction, myocardial infarction, peripheral vascular disease, asthma, bronchospastic lung disease, chronic obstructive lung disease, gastrointestinal disorders, hypercoagulation states, thrombocytosis, eclampsia, or pre- eclampsia.

12. The method of claim 9, further comprising providing an additional pharmaceutical composition to said patient.

13. The method of claim 12, wherein said additional pharmaceutical composition is selected from the group consisting of “beta blockers,” anti-hypertensives, cardiotonics, anti-thrombotics, vasodilators, hormone antagonists, endothelin receptor antagonists, cytokine inhibitors/blockers, calcium channel blockers, other phosphodiesterase inhibitors, and angiotensin type 2 antagonists.

14. The method of claim 13, wherein said endothelin receptor antagonist is ambrisentan.

15. The method of claim 12, wherein said endothelin receptor antagonist is darusentan.
